(19) United States
(12) Patent Application Publication
(10) Pub. No.: US 2007/0048873 A1
(43) Pub. Date: Mar. 1, 2007

(54) Method of quantitative analysis of hexavalent chromium in chromate coating

(75) Inventors: Tetsuya Tachibe, Kawasaki-shi (JP); Mitsuhiro Oki, Yokohama-shi (JP); Miyuki Takenaka, Yokohama-shi (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: Kabushiki Kaisha Toshiba, Minato-ku (JP)

(21) Appl. No.: 11/511,331

(22) Filed: Aug. 29, 2006

(30) Foreign Application Priority Data
Sep. 1, 2005 (JP) 2005-253015

(51) Int. Cl. G01N 33/20

(52) U.S. Cl. 436/83

(57) ABSTRACT

A method of quantitative analysis of hexavalent chromium in a chromate coating includes immersing a substrate on which a chromate coating is formed in an aqueous solution containing lithium hydroxide to extract hexavalent chromium, and performing quantitative analysis of extracted hexavalent chromium.

### CROSS-REFERENCE TO RELATED APPLICATIONS

This application is based upon and claims the benefit of priority from prior Japanese Patent Application No. 2005-253015, filed Sep. 1, 2005, the entire contents of which are incorporated herein by reference.

### BACKGROUND OF THE INVENTION

1. Field of the Invention

The present invention relates to a method of quantitative analysis of hexavalent chromium in a coating for a metal substrate such as a chromate coating used in household electronic appliances and automobiles.

2. Description of the Related Art

In Europe, it is required to restrict the use of lead, mercury, cadmium, PBB, PBDE, and hexavalent chromium in principle in accordance with the Restriction of Hazardous Substances Directive (ROHS) which takes effect on Jul. 1, 2006. In order to comply with the directive, it is desired to develop analytical methods capable of conveniently assaying these substances.

It is known that X-ray photoelectron spectroscopy can be used as an analytical method for directly assaying hexavalent chromium content in a chromate coating (see Jpn. Pat. Appln. KOKAI No. H05-164710). Since this method assays only the surface of the coating, however, there are problems that the analytical value does not represent the value for the entire sample and that it is hard to separate peaks of trivalent chromium and hexavalent chromium.

ISO 3613 defines an analytical method for determining hexavalent chromium eluted from a chromate coating using boiling water. This method assays only hexavalent chromium eluted by the boiling water, and cannot determine total hexavalent chromium content present in the chromate coating.

An analytical method using sodium hydroxide solution as an extracting solution is known in “Method 3060” defined by the United States Environmental Protection Agency (EPA). When a chromate coating on a substrate of an amphoteric metal such as aluminum is assayed using the extracting solution, the aluminum substrate is eluted prior to the chromate coating accompanied by hydrogen gas generation and reduction of hexavalent chromium to trivalent chromium, making it difficult to perform highly accurate quantitative analysis of hexavalent chromium.

Further, as an analytical method of efficiently extracting hexavalent chromium in a chromate coating on a metal substrate in a short time, a method is known in which the chromate coating is cracked and then the coating is immersed in an extracting solution to extract hexavalent chromium so as to be analyzed (see Jpn. Pat. Appln. KOKAI No. 2004-325321). Since this method requires to applying thermal shock or mechanical shock to the substrate in order to cause cracks, however, there is a possibility that the substrate itself may be broken. Thus, this method cannot be generally used for analyzing hexavalent chromium.

### BRIEF SUMMARY OF THE INVENTION

According to an aspect of the present invention, there is provided a method of quantitative analysis of hexavalent chromium in a chromate coating, comprising: immersing a substrate on which a chromate coating is formed in an aqueous solution containing lithium hydroxide to extract hexavalent chromium; and performing quantitative analysis of extracted hexavalent chromium.

### BRIEF DESCRIPTION OF THE SEVERAL VIEWS OF THE DRAWING

FIG. 1 is a graph showing a relationship between an extraction time and an amount of extracted chromium when an aluminum substrate having a chromate coating is immersed in different extracting solutions; and

FIG. 2 is a graph showing a relationship between an extraction time and an amount of eluded aluminum when an aluminum substrate having a chromate coating is immersed in different extracting solutions.

### DETAILED DESCRIPTION OF THE INVENTION

Embodiments of the present invention will be described below.

Chromating is carried out to improve corrosion resistance and coating performance of a metal substrate. The chromating is generally performed by immersing a metal substrate liable to be corroded in a chromic acid solution to form a chromate coating. The chromate coating includes those types called bright chromate, colored chromate, black chromate, and green chromate depending on a thickness, chromium content, other element content, and so forth. The present invention can be applied to any type of chromate.

The chromating method includes reactive chromating, application chromating, and electrolytic chromating. The chromating method is not particularly limited, and the present invention can be applied to a coating formed by any type of chromating.

A metal substrate used is not particularly limited. The metal substrate includes, for example, a zinc-plated steel substrate for improving corrosion protection, the surface of which is chromated. The metal substrate also includes an aluminum substrate liable to be corroded, the surface of which is chromated.

According to embodiments of the present invention, an aqueous solution containing lithium hydroxide is used as an extracting solution. If an extracting solution containing sodium hydroxide is used and the extracting solution is applied to an aluminum substrate, the sodium hydroxide corrodes the aluminum substrate, making it impossible to expect an accurate analytical result. In contrast, the aqueous solution containing lithium hydroxide does not corrode the aluminum substrate. Thus, the method according to embodiments of the present invention can be applied to any metal substrate.

In an embodiment of the present invention, a concentration of the lithium hydroxide in the extracting solution is preferably in a range of 0.8 to 1.3 wt %, and more preferably in a range of 0.9 to 1.1 wt %. If the concentration of the lithium hydroxide in the extracting solution is less than 0.8 wt %, the reaction of the lithium hydroxide with the chromate coating proceeds slowly, leading to disadvantageously long extraction time. If the concentration of the lithium hydroxide in the extracting solution exceeds 1.3 wt %, the lithium hydroxide reacts with the chromate coating intensively, and thus there is a disadvantageous possibility of reducing the hexavalent chromium.

Incidentally, it is preferable in the embodiments of the present invention to use lithium hydroxide with purity of reagent grade. However, the lithium hydroxide may contain another component such as potassium hydroxide as long as the component does not exert such an adverse effect of corroding the substrate or reducing haxavalent chromium.

It is preferable to set the temperature of the extracting solution to a range of 60 to 90° C. If the temperature of the extracting solution is lower than 60° C., the reaction of the lithium hydroxide with the chromate coating proceeds slowly, leading to disadvantageously long extraction time. If the temperature of the extracting solution exceeds 90° C., the lithium hydroxide reacts with the chromate coating intensively, and thus there is a disadvantageous possibility to reduce the hexavalent chromium.

The thus extracted hexavalent chromium can be assayed by general, high-precision quantitative analysis such as spectrophotometry using diphenylcarbazide.

### EXAMPLES

The present invention will be described in more detail based on examples.

(Substrate)

An aluminum plate (A5052), 20 mm×20 mm×0.5 mm in thickness, having a chromate coating with a thickness of 250 nm was used as a substrate.

(Extraction)

The sample was placed in a 30-mL glass beaker to which an extracting solution was added so as to immerse the sample in the extracting solution. As the extracting solution, a 1 wt % aqueous solution of lithium hydroxide (extracting solution 1, Example), a 1 wt % aqueous solution of sodium hydroxide (extracting solution 2, Comparative Example), or a mixed aqueous solution of sodium hydroxide and sodium carbonate (extracting solution 3, Comparative Example) was used. The beaker was placed on a hot plate. The extracting solution was heated over a predetermined period of time to extract hexavalent chromium in the chromate coating.

(Quantitative Analysis)

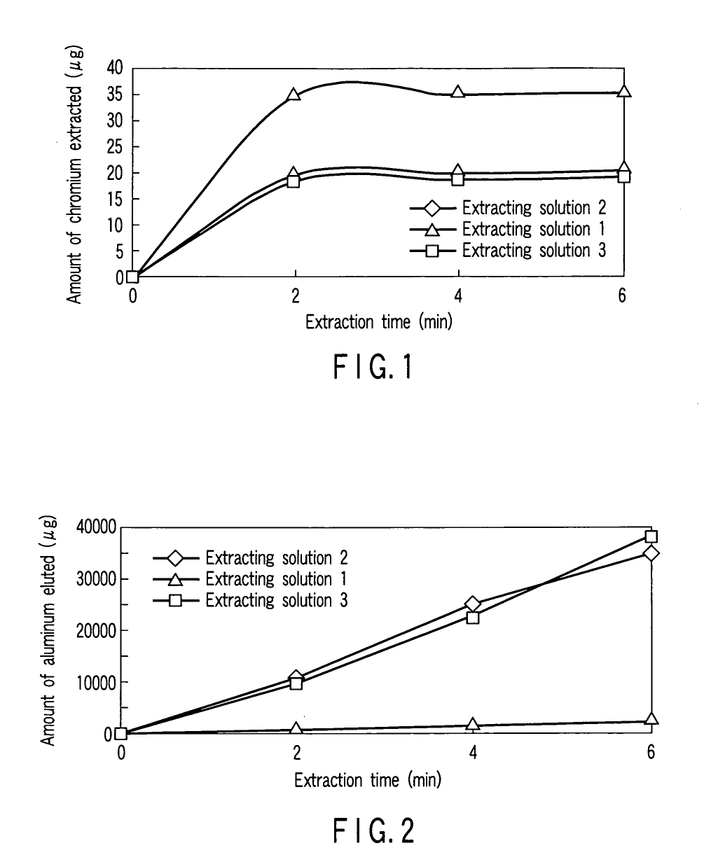

The resultant hexavalent chromium extract was allowed to natural cooling, and then transferred to a 50-mL volumetric flask to which 2 mL of 25% sulfuric acid was added to make the extract acidic. Then, quantitative analysis of hexavalent chromium was performed by the official method of spectrophotometry using diphenylcarbazide. FIG. 1 shows the results. FIG. 1 is a graph showing a relationship between an extraction time and an amount of extracted chromium for different extracting solutions.

An amount of aluminum eluted from the substrate was also measured. FIG. 2 shows the results. FIG. 2 is a graph showing a relationship between an extraction time and an amount of eluded aluminum for different extracting solutions.

As is shown in FIG. 1, the amounts of extracted chromium are: 35 μg for the 1 wt % aqueous solution of lithium hydroxide (extracting solution 1), 20 μg for the 1 wt % aqueous solution of sodium hydroxide (extracting solution 2), and 19 μg for the mixed aqueous solution of sodium hydroxide and sodium carbonate (extracting solution 3). It is found that, when the 1 wt % aqueous solution of sodium hydroxide (extracting solution 2) or the mixed aqueous solution of sodium hydroxide and sodium carbonate (extracting solution 3) is used, 40% of hexavalent chromium is reduced compared with that extracted when the 1 wt % aqueous solution of lithium hydroxide (extracting solution 1) is used.

It is found that, when the 1 wt % aqueous solution of lithium hydroxide (extracting solution 1) is used, the amount of aluminum eluted from the substrate is very small compared with that eluted when the 1 wt % aqueous solution of sodium hydroxide (extracting solution 2) or the mixed aqueous solution of sodium hydroxide and sodium carbonate (extracting solution 3) is used.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A method of quantitative analysis of hexavalent chromium in a chromate coating, comprising:
immersing a substrate on which a chromate coating is formed in an aqueous solution containing lithium hydroxide to extract hexavalent chromium; and
performing quantitative analysis of extracted hexavalent chromium.

2. The method according to claim 1, wherein the lithium hydroxide is contained in the solution at a concentration of 0.8 to 1.3 wt %.

3. The method according to claim 2, wherein the lithium hydroxide is contained in the solution at a concentration of 0.9 to 1.1 wt %.

4. The method according to claim 1, wherein the substrate is an aluminum substrate.

5. The method according to claim 1, wherein the substrate is a zinc-plated steel substrate.

6. The method according to claim 1, wherein the solution is 60 to 90° C.

7. The method according to claim 1, wherein the substrate is immersed in the solution for 3 to 5 minutes.

8. The method according to claim 1, wherein the extracted chromium is subjected to quantitative analysis by spectrophotometry using diphenylcarbazide.
